(19) United States
(12) Patent Application Publication
(10) Pub. No.: US 2019/0231986 A1
(43) Pub. Date: Aug. 1, 2019

(54) In-Line Filter For Protein/Peptide Drug Administration

(71) Applicant: Lupin Limited, Mumbai (IN)

(72) Inventors: Prasanna Kumar Devaraneni, Pune (IN); Rustom Sorab Mody, Pune (IN)

(21) Appl. No.: 16/330,125

(22) PCT Filed: Sep. 19, 2017
(86) PCT No.: PCT/IB2017/055656

§ 371 Date: Mar. 4, 2019

(30) Foreign Application Priority Data
Sep. 19, 2016 (IN) 201621031927

(51) Int. Cl. A61M 5/31; C07K 16/22; B01D 35/02; B01D 39/16; B01D 39/18

(52) U.S. Cl. CPC A61M 5/3145; C07K 16/22; B01D 35/02; B01D 2201/184; B01D 39/18; A61M 2205/7545; C07K 2317/24; B01D 39/16

(57) ABSTRACT

The present invention relates to incorporation of in-line filter into the drug administration device to minimize the entry of particulates into the human body during injection of therapeutic proteins/peptides. Particulate matter can be of non-proteinaceous and/or proteinaceous and/or mixture thereof. Particles such as undissolved or precipitated solids, fibers, glass flakes, rubber fragments, silicone oil etc. represent non proteinaceous particles while protein aggregates (amorphous and fibrils) represent proteinaceous particles. Although particulate matter in injectable formulation require to be controlled within various regulatory and compendial limits, methods to minimize particulate matter further are beneficial as proteinaceous particulates poses the risk of immunogenicity.

### FIELD OF THE INVENTION

The present invention relates to incorporation of one or more in-line filter/s into the drug administration device and the use of such device for administration of therapeutic protein/peptide drug. Further the use of in-line filter would minimize adverse reactions associated with particulate matter especially immunogenic reactions.

### BACKGROUND OF INVENTION

Protein/peptide drug play an important role in the treatment of various discuses. Most of these therapeutic proteins/peptides are delivered via parenteral route. Hence, one major aspect is that these drugs should be practically free from any particulate matter.

Particulate matter in parenteral drug product consists of extraneous mobile undissolved particles, other than gas bubbles, unintentionally present in solutions. The typical sources of particulate matter are environment, packaging materials, formulation components, active principal, product packaging interactions and process-generated particles. The most commonly observed non-proteinaceous particles in protein formulations are silicone oil, cellulose fibers, cotton, glass micro flakes, rubber, plastic or metal while protein aggregates represent proteinaceous particles.

Using combination of chromatographic and filtration methods, downstream processing keeps the particulate count low. However during formulation and filling process, multiple unit operations may contribute to additional particulates which again con be controlled by suitable final filtration step before till finish operation. However as a result of multiple stresses, particulate matter can be generated from primary container closure and drug product during shelf life. Particles generated during shelf life could range from sub-visible to visible range and accordingly different methods of analysis have been recommended.

Particulate matter can be harmful when introduced into the bloodstream. Several reports describe adverse impact on organs like eyes, brain, lungs, heart, kidney, spleen, stomach and intestine. These particles arc reported to cause mechanical blockage of arterioles and capillaries, activation of platelets, neutrophils and/or endothelial cells with a subsequent generation of occlusive micro-thrombi and granuloma.

Unlike non-proteinaceous particles, protein based particles (aggregates) are thought to cause immunogenic reactions, typically involving the formation of neutralizing antibodies that decrease physiologically effective concentration of the therapeutic drug and triggering severe allergic responses like anaphylaxis or serum sickness. A well reported example of a severe immunogenic reaction is the pure red cell aplasia, resulting from the formation of anti-erythropoietin antibodies. Protein aggregates (particles) may also cause an immune response via T cell wherein T cells recognize repetitive patterns on the surface of aggregates which are similar to the unique epitope arrangement of microbial antigens.

Factors like temperature, pH, shaking, shearing are considered to be major reasons for the formation of protein aggregates. Silicone oil used as lubricant in glass syringes, vial and syringe stoppers plus the material of stoppers is also reported to induce protein aggregation/particle formation. In addition, factors like accidental freeze thaw, exposure to light might also contribute to proteinaceous particle generation. Above factors in an unforeseen combination can exaggerate particle generation.

Protein engineering and formulation Optimization have been adopted to reduce the immunogenicity of proteins by minimizing aggregation propensity. Additionally, silicone oil based particles can be controlled by use of baked-on process for silicone oil lubrication onto glass syringe or use of silicone oil free plastic syringe. However, it is not clear if such approaches will completely prevent introduction or generation or protein and non-protein based particulates during the filling and shelf-life storage of protein injectable.

Another solution that is widely practiced to overcome negative aspects associated with the particles is use of filter in needles having larger bore. Such needles are specifically used for withdrawal of drug solution from the vial. These type of needles with large bores are usually referred to as blunt filter needles and are available in the market. However in practice the blunt needles needs to be replaced with administration needles prior to injecting the drug solution. This practice of changing needles prior to administration increases the chances of contamination and also some amount of drug is lost due to such practice which makes Otis method economically unviable. Further such approach is unsuitable with prefilled syringes where chances of particle contamination is higher.

US20100111963 discloses use of ranibizumab for treatment of age related macular degeneration. In its disclosure use of filter needle for drug withdrawal is described wherein 0.23 ml ranibizumab dose solution is withdrawn through a 5 μm filter needle. The filter needle is removed and replaced with a 30-gauge, ½ inch Precision Glide® needle, and excess ranibizumab is expelled and then the drug is injected intra-vitreally. One drawback of such method is that although the dose solution is filtered while withdrawal from vial, the silicone used in administration syringes may shred and add to the particle count which may pose immunogenic risk to the patients. Also, as previously mentioned, such practice increases the chances of contamination and also some amount of drug is lost due to such practice which makes this method economically unviable. Further this approach is unsuitable with prefilled syringes where chances of particle contamination is higher.

US20150258280 discloses use of filter for installation into the syringe prior to drug administration. The disclosure specifically focuses on use of filter for administration of analgesics. However the disclosure is silent about the use of the filter for administration of protein/peptide drugs which are more prone to contamination and are more costly as compared to synthetic analgesics.

WO9808561 discloses use of aseptized cotton incorporated in the flare of the syringe for discharging liquid medicinal product. However use of cotton with protein/peptide may pose additional risk and may also lead to loss of costly therapeutic protein due to absorption/adsorption and hence may not be economically viable.

Hence there is lack of effective methods to minimize the particulate matter during injection of drug solution to the patient without compromising sterility of the drug product. Any such method to minimize the proteinaceous and/or non-proteinaceous particulates may reduce the risk associated with immunogenicity.

### SUMMARY OF THE INVENTION

The present invention describes the use of drug administration device with an in-line filter to reduce the particulate matter so that the drug product would enter into human body directly post filtration without any need of further additional steps. Such in-line filter would minimize the particle count that could potentially be immunogenic to human. The present inventors have surprisingly found that the use of in-line filters reduces the number of particles that could be potentially immunogenic in nature. The immunogenic reactions of drug delivered through in-line filter would thus be significantly lower as compared to non-filtered drug. Finally the forces required for injection of the drug solution from the syringe with in-line filter of the present invention are comparable to the forces required for injection from a syringe without filter. The in-liner filter of the present invention therefore overcome all the encountered problems exemplified above and may be conveniently used for the administration of protein/peptide drugs.

### OBJECTIVES OF INVENTION

The main objective of the invention is to use in-line filter into the drug administration device to minimize the entry of particulates into the human body during injection of therapeutic proteins/peptides. Use of in fine filer would minimize adverse reaction associated with particulate matter especially immunogenic reaction.

Another objective of the present invention is to provide in-line filter with drug administration devices comprising but not limited to disposable syringe, lubricated syringes, prefilled syringes, auto injector, prefilled pen and other delivery devices.

Yet another objective of the present invention is to provide in-line filter into the drug administration device prior to administration of drug so as to provide the medicament with reduced immunogenicity.

Yet another objective of the present invention is to provide in-line filter into the drug administration device to minimize the particulates which may pose risk of immunogenicity to the human body.

Yet another objective of the present invention is lo provide in-line filter into the drug administration device to minimize the particulates which may pose risk of immunogenicity to the human body without undue increase in the gliding or instantaneous force.

Yet another objective of the present invention is to provide in-line filter into the drug administration device with zero or substantially less protein binding.

In accordance with the principle of the present invention, not only is contamination minimized by filtering the liquid as it is being injected, but the present invention also eliminates the need of replacing needles between the withdrawal and injection steps. As a result, user have to employ fewer or rather no manipulative steps by the use of drug administration device with in-line filter of the present invention.

Overall, use of the in-line filter of the present invention provides a simplified procedure for administration of protein/peptide therapeutics, without compromising the sterility of the formulation and additionally reducing the risk associated with the entry of particulates into the human body.

The details of one or more embodiments of the invention are set forth in the description below. Other features, objects and advantages of the invention will be apparent from the following description including claims.

### BRIEF DESCRIPTION OF THE FIGURES

FIG. 1 shows a side view of o syringe and its components

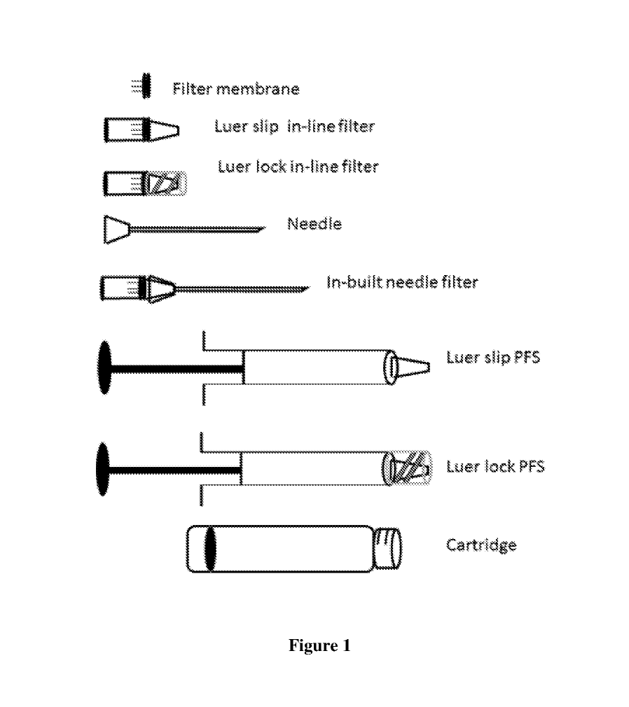

FIG. 2 shows the hold-up volume of in-line syringe filters

### DETAILED DESCRIPTION OK THE INVENTION

The present invention as illustratively described in the following may suitably be practiced in the absence of any element or elements, limitation or limitations, not specifically disclosed herein.

Those skilled in the art will recognize or be able to ascertain using no more than routine experimentation many equivalents to the specific embodiments described herein. The scope of the present invention is not intended to be limited to the description, but rather is as set forth in the appended claims.

The in-line filter causes subsequent reduction of particulate load post filtration of therapeutic proteins. Such reduction of particulates would depend on the cut off (pore size) of membrane filter. The area of in-line filter should be small enough to reduce the panicles without significant impact on gliding forces. Ideal filter should have low hold-up volume and minimal loss of non-aggregated protein with maximum retention of particulates (proteinaceous and non-proteinaceous).

The term “about” or “approximately” can mean within an acceptable error range for the particular value as determined by one of ordinary skill in the art which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%. up to 5%, or up to 1% of a given value.

Substantially free may include containing less than 5% of said particles, particularly less than 1%, for example less than 0.5%, such as less than 0.1%.

“Administration” is given its ordinary and customary meaning of delivery by any suitable means recognized in the art. Exemplary forms of administration include oral delivery, anal delivery, direct puncture or injection, including intravenous, intraperitoneal, intramuscular, subcutaneous, intratumoral, intravitreal and other forms of injection, gel or fluid application to an eye, ear, nose, mouth, anus or urethral opening not involving a solid-state carrier such as a microsphere or bead, and cannulation. A preferred mode of administration is injection by syringe, typically a needle-bearing syringe.

The term “treatment” or “treating” includes the administration, to a subject in need, of an amount of a compound that will inhibit, decrease or reverse development of a pathological condition.

A “dose administration device” is a device for providing a substance, such as a protein/peptide therapeutic, to a subject such as an animal or human patient. Dose administration device generally contain the substance, such as a protein/peptide, and also provide the capacity to discharge the substance. The present invention is generally embodied in a syringe set as an in-line filter for removing any microscopic particulate from the fluid stream as it is administered to the patient. Other dose administration devices include, but are not limited to, syringes comprising at least one chamber and infusion modules comprising at least one chamber. In a preferred embodiment the drug administration device comprises but are not limited to disposable syringe, prefilled syringes, auto injector, prefilled pen and other delivery devices.

A “pre-filled syringe” is a syringe which is supplied by the drug manufacturer in a filled state, i.e. a measured dose of the drug to be administered is already present in the syringe when it is purchased and ready for administration. In particular, the pharmaceutical composition containing the drug does not have to be drawn from a vial containing the composition by using an empty syringe.

The “particulates” can be defined as particulate matter which may be non-proteinaceous and/or proteinaceous and/or mixture thereof. Particles such as undissolved or precipitated solids, fibers, glass flakes, rubber fragments, silicone oil etc. represent non-proteinaceous particles while protein aggregates (amorphous and fibrils) represent proteinaceous particles.

The in-line filter of the foregoing embodiments may be in any suitable form preferably in the form of membrane or as microporous hollow fibers most preferably in the form of depth filters or nubs.

The in-line filter in all the foregoing embodiments may be formed of any appropriate material, such as but not limited to cellulose acetate, cellulose mixed ester (acetate and nitrate), regenerated cellulose, glass microfiber, nylon, polymide 6, polyethersulphone (PES), polypropylene (PP), polytetrafluoroethylene (PTFE), polyvinylidene fluoride (PVDF) or perfluoropolyether (PFPE). The other component parts of the filters may also be formed of any appropriate materials such as those known in the prior art.

The in-line filter may be used with needle sizes comprising but not limited to 30 gauge×½ inch, 27, 31, 32, 33 or 34 gauge needle.

The in-line filter of the present invention has a pore size in the range of but not limited to 0.1-10.0 μm.

The syringe has a nominal fill volume, i.e. a volume which can be maximally taken up by the syringe of 0.05 ml to 1.5 ml preferably, and most preferably 0.2 ml to 1.0 ml.

The skilled person typically knows that there is a hold up volume of drug product due to the dead space within the syringe, needle and the loss during the preparation of the syringe for injection. Hence the syringe is usually filled with a product volume which is larger than the deliverable volume.

The in-line filter described above are preferably inserted into the syringes during manufacture thereof and can thus be sterilized in-situ by known methods. However, it may be appropriate in some situations for the filters to be supplied separately for subsequent fitting.

The in-line filter of the present invention may be used with any pharmaceutical and/or biotechnological molecules preferably it can be used for therapeutic protein/peptide comprising of but not limited to Fc fusion proteins, monoclonal antibodies, Fab fragments, growth factors most preferably for VEGF antagonists.

The term “VEGF antagonist” refers to a molecule which specifically interacts with VEGF and inhibits one or more of its biological activities, e.g. its mitogenic, angiogenic and/or vascular permeability activity. It is intended to include both anti-VEGF antibodies and antigen-binding fragments thereof and non-antibody VEGF antagonists.

The term “anti-VEGF antibody” refers to an antibody or antibody fragment such as a Fab or a scFV fragment that specifically binds to VEGF and inhibits one or more of its biological activities, e.g. its mitogenic, angiogenic and/or vascular permeability activity. Anti-VEGF antibodies act. e.g., by interfering with the binding of VEGF to a cellular receptor, by interfering with vascular endothelial cell activation after VEGF binding to a cellular receptor, or by killing cells activated by VEGF. Anti-VEGF antibodies include, e.g., antibodies A4.6.1, bevacizumab, ranibizurmib, G6, B20, 2C3, and others as described in, for example. WO 98/45331. US 2003-0190317, U.S. Pat. Nos. 6,582,959, 6703020, WO 98/45332, WO 9030046, WO 94/10202, WO 2005/044853, EP 0666868. WO 2009/155724 and Popkov et al. (2004) J. Immunol. Meth. 288: 149-64.

Preferably, the anti-VEGF antibody or antigen-binding fragment thereof present in the pharmaceutical composition of the present invention is ranibizumab or bevacizumab or aflibercept. Most preferably, it is ranibizumab or an antigen-binding fragment thereof.

The use of in-line filter of the present invention is preferably for but not limited to administration of VEGF antagonist to a patient having ocular diseases, preferably having an ocular disease selected from the group consisting of age-related macular degeneration (AMD), visual impairment due to diabetic macular oedema (DME), visual impairment due lo macular edema secondary to retinal vein occlusion (branch RVO or central RVO), diabetic retinopathy in patients with diabetic macular edema or visual impairment due to choroidal neovascularization (CNV) secondary to pathologic myopia.

The syringe with in-line filter of the present invention provides formulation with low particulate count. The % reduction in amount of visible particles in the contained formulation post filtration, determined by conventional means, is most preferably 100%. The % reduction in amount of sub-visible particles (2-50 μm) by use of in-line filter of the present invention is preferably in the range of 99-100%, more preferably in the range of 60-70% and most preferably in the range of 85-95%. The in-line filter of the present invention causes a % reduction in number of particles of size 0.2-50 μm preferably within the range of 50% to 70% most preferably in the range of 80-95%.

The syringe with in-line filter of the present invention further has excellent gliding behavior. In particular, the instantaneous force, i.e. the force required to initiate the movement of the plunger, is less than 15N or 12N, preferably less than 10N or 9N, more preferably less than 6N and most preferably less than 5N.

Further, the gliding force, i.e. the force required to sustain the movement of the plunger along the syringe barrel to expel the liquid composition, is less than 15N, preferably less than 12N, more preferably less than 10N and most preferably less than 7N. In a particularly preferred embodiment there is no significant difference between the instantaneous force and the gliding force.

The in-line filter of the present invention has very low or zero protein binding. Binding can be defined as the property of the protein/peptide formulation to have an affinity for filter media or other filter components. The amount of protein bound to the in-line filter of the present invention, measured by conventional methods, is preferably 0.1% and most preferably the protein binding to the in-line filter is zero.

Further, the in-line filter of the present invention has zero or minimum extractables and leachables. Extractables are defined as chemical entities, both organic and inorganic, that will potentially extract from components of a filter or device into the drug product under accelerated conditions. Leachables are chemical entities, both organic and inorganic, that migrate from components of a container closure system or device or filter into a drug product over the course of its shelf-life. Minimum in the context of the present invention can be defined as being within various regulatory and compendial limits.

The present Invention has been described in terms of the preferred embodiment for the purpose of illustration and not limitation, it is intended to include those equivalent structures, some of which may be apparent upon reading this description, and others that may be obvious only after some study.

### EXAMPLES

### Example 1: Comparison of Reduction in Total Particulate Count using Needle with In-Line Filter

Ranibizumab binds to VEGF and prevents VEGF interaction with cognate receptors. Ranibizumab is Fab fragment designed for intravitreal injection to treat macular degeneration. Ranibizumab drug substance in formulation buffer was subjected to UV exposure for 3 hours to generate proteinaceous particles and filled into Pre-filled Syringe (PFS) of different make coated with different levels of silicone oil. After overnight incubation at room temperature, PFS contents were emptied manually with or without in-line filtration in a Class 100 environment. Particle count was measured using Light obscuration (LO) spectroscopy. For comparative purpose, here we used two different makes of PFS and 3 different makes of in-line filters of which one filter was in-line with needle (needle with built in filter).

Result: Contents from the PFS was emptied into a clean container in a laminar flow hood (Class 100 workstation) after attaching needles that were with and without in-line filters (unfiltered). The ejected liquid was measured for particle counts using LO. Total number of particles observed in an unfiltered condition was considered as 100% and relative reduction of total number of particles was calculated for different filters used. Results shown in Table 1, indicates that all three filters showed significant reduction in total particle count of greater than 2 μm size. However, the % reduction in die particle count was also dependent on the make of PFS. Hence development of optimal combination of PFS and filter is critical to keep the total particle count low.

TABLE 1
Comparison of reduction in total particulate count
using needle with in-line filter.

% Reduction in no. of
particles of > 2 μm size

| Sample details | PFS-A | PFS-B |
|---|---|---|
| PFS fitted with Filter-1 | 57.3 | 83.5 |
| PFS fitted with Filter-2 | 66.6 | 87.4 |
| PFS fitted with Filter-3* | 91.2 | 86.0 |

*Needle with in-line filter

### Example 2: Evaluating the Efficacy of In-Line Syringe (Liters in Removing Silicone Oil Droplets

The efficacy of in-line syringe filters to capture silicone oil particles was tested with a 200 μg/ml silicone oil emulsion challenge test. In this study, 200 μg/ml silicone oil emulsion was prepared in Ranibizumab formulation buffer, 1 ml of which was aspirated in 1 ml Tuberculin syringe. The syringe was attached to 0.45 μm cut-off in-line PVDF/PES syringe filter and the contents emptied into clean Eppendorf tubes. Silicone oil emulsion (SOE), and samples through the in-line syringe filters were analyzed for sub-visible particulate matter by MicroFlow Imaging (MFI).

Particle concentration in cumulative size bins ≥5 μm, ≥10 μm, ≥25 μm and ≥50 μm are reported in this study.

Result: It was observed that 0.45 μm PVDF in-line syringe filters efficaciously captured silicone oil particles and caused a significant reduction of silicone oil particles present in original samples containing 200 μg/ml silicone oil emulsion. A similar observation was observed with silicone oil emulsion samples filtered with 0.45 μm PES in-line syringe filters. A significant reduction in the sub-visible panicle counts was observed in cumulative size bins ≥5 μm, ≥10 μm, ≥25 μm and ≥50 μm.

TABLE 2
Sub-visible particle counts of silicone oil emulsion in
Ranibizumab formulation buffer passed through siliconized
prefilled syringe with/without in-line syringe filter.

| | Particle Concentration (#/ml) | | | | | | | |
|---|---|---|---|---|---|---|---|---|
| | ≥5 μm | | ≥10 μm | | ≥25 μm | | ≥50 μm | |
| Sample | Mean | SD | Mean | SD | Mean | SD | Mean | SD |
| SOE | 38744 | 7726 | 3095 | 1617 | 97 | 18 | 3 | 2 |
| SOE through | 40 | 27 | 12 | 4 | 4 | 2 | 0 | 1 |
| 0.45 μm | | | | | | | | |
| PVDF filter | | | | | | | | |
| % Reduction | 99.9 | | 99.6 | | 95.8 | | 100 | |
| of particulates | | | | | | | | |

### Example 3: Evaluating the Efficacy of In-Line Syringe Filters in Removing Sub-Visible Ranibizumab Aggregates

In this study, the efficacy of 0.45 μm cut-off in-line PVDF in-line syringe filter in capturing sub-visible Ranibizumab aggregates were evaluated. Ranibizumab Drug Product (0.23 ml in vial) was incubated at 70° C. for 6 hours to generate sub-visible aggregate. Then the contents of three vials were pooled and aspirated into siliconized prefillable syringe. The in-line syringe filter was then connected to 30G×½″ needle and the content emptied into clean Eppendorf tubes. Aggregated Ranibizumab samples and filtered aggregated Ranibizumab samples in addition to control unstressed Ranibizumab drug product were tested for particulate matter by MFI.

Result: It was observed that 0.45 μm PVDF in-line syringe filters significantly reduced the concentration of sub-visible particles in cumulative size bins ≥5 μm, ≥10 μm and ≥25 μm. Sub-visible particles ≥50 μm observed in heat stressed Ranibizumab samples was compared to unstressed Ranibizumab control.

TABLE 3
Sub-visible concentration of Ranibizumab DP control, heat stressed
Ranibizumab and heat stressed Ranibizumab through siliconized syringe
in the presence and absence of in-line syringe filter

| | ≥5 μm | | ≥10 μm | | ≥25 μm | | ≥50 μm | |
|---|---|---|---|---|---|---|---|---|
| Sample | Mean | SD | Mean | SD | Mean | SD | Mean | SD |
| Ranibizumab | 459 | 111 | 109 | 25 | 11 | 5 | 2 | 2 |
| DP Control | | | | | | | | |
| Ranibizumab DP | 1405 | 61 | 552 | 52 | 78 | 22 | 4 | 4 |
| Heat Stressed | | | | | | | | |
| 0.45 μm filter | 193 | 49 | 50 | 18 | 1 | 2 | 0 | 0 |
| % Reduction | 86.2 | | 90.9 | | 98.7 | | 100 | |
| of particulates | | | | | | | | |
| compared to DP | | | | | | | | |
| Heat Stressed | | | | | | | | |

### Example 4: Evaluating the Efficacy of In-Line Syringe Filters in Removing from Ranibizumab containing Sub-Visible Aggregates and Silicone Oil Droplets

In this study, the efficacy of either 0.45 μm cut-off in-line PVDF in-line syringe filter in capturing sub-visible Ranibizumab aggregates and silicone oil droplets were evaluated. Ranibizumab Drug Product (0.23 ml in vial) was incubated at 70° C. for 6 hours to generate sub-visible aggregates. Then the contents of three vials were pooled and spiked with silicone oil emulsion such that the final concentration of silicone oil in the sample was 100 μg/ml. Approximate 500 μL of this sample was aspirated into siliconized prefillable syringe. The in-line syringe filter was then connected to 30G×½″ needle and the content emptied into clean Eppendorf tubes. Aggregated Ranibizumab samples containing silicone oil and filtered Ranibizumab samples were tested for particulate matter by MFI.

Result: It was observed that 0.45 μm cutoff filters were both efficient in capturing sub-visible Ranibizumab aggregates and silicone oil. Reduction in sub-visible particles was observed in cumulative size bins ≥5 μm, ≥10 μm, ≥25 μm and ≥50 μm.

TABLE 4
Table showing the sub-visible concentration of aggregated Ranibizumab containing spiked
silicone oil emulsion, and same samples filtered through 0.45 μm in-line syringe filters

| | ≥2 μm | | ≥5 μm | | ≥10 μm | | ≥25 μm | | ≥50 μm | |
|---|---|---|---|---|---|---|---|---|---|---|
| Sample | Mean | SD | Mean | SD | Mean | SD | Mean | SD | Mean | SD |
| Ranibizumab | 67918 | 15677 | 16871 | 5475 | 4107 | 2009 | 497 | 400 | 145 | 162 |
| Aggregate + SOE | | | | | | | | | | |
| 0.45 μm filtered | 521 | 168 | 92 | 17 | 24 | 14 | 3 | 2 | 1 | 2 |
| % Reduction of | 99.23 | | 99.45 | | 99.41 | | 99.39 | | 99.31 | |
| particulates | | | | | | | | | | |

*SOE is silicone oil emulsion

### Example 5: Evaluating Adsorption of Ranibizumab on In-Line Syringe Filter

In this study four concentrations of Ranibizumab ranging from high to low concentration were chosen for analysis 10 mg/ml, 5 mg/ml, 1 mg/ml and 0.6 mg/ml. Then 0.165 ml of Ranibizumab was aspirated in prefillable syringe, attached to a 0.45 μm in-line syringe filter and contents emptied into a clean centrifuge tube. As a control 0.163 ml of Ranibizumab was aspirated into prefillable syringe and contents emptied into centrifuge tubes. The concentration of Ranibizumab samples in the centrifuge tubes were determined assuming ϵ₂₈₀ nm^{mg/mL}=1.8. Ranibizumab concentration in control and filtered samples were compared.

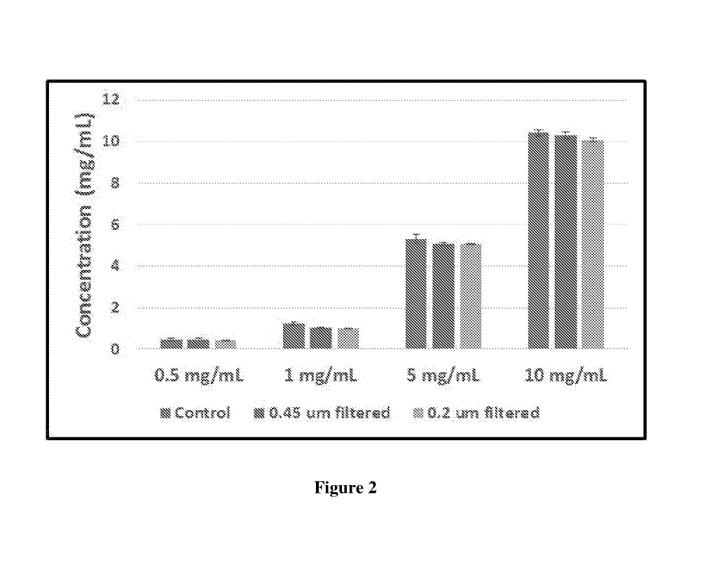

Result: The results of the analysis is shown in FIG. 2. Overall, the concentration of Ranibizumab control and samples passed through filter remained comparable and drastic loss of Ranibizumab due to adsorption on in-line filters was not observed.

### Example 6: Determination of the Hold-Up Volume of In-Line Syringe Filters

The hold-up volume of in-line syringe filters was determined by a gravimetric method. First the dry weight of the in-line syringe filter is measured in an analytical balance. Then 0.5 ml of Ranibizumab formulation buffer was aspirated in the prefillable syringe. The syringe filled with formulation buffer was connected to either a 0.45 μm or 0.2 μm in-line syringe filter and contents emptied. The in-line syringe filter was then detached and the weight of the wet filter measured. The volume of buffer in the syringe filter was determined from the following equation.

$$\frac{Weight of Wet filter - Weight of Dry filter}{Density of Buffer}$$

Result: The mean hold-up volume of the in-line syringe tillers approximately 62 μl in case of PVDF 0.45 μm filter and approximately 71 μl in the ease of PVDF 0.2 μm filter. Overall it was found that the hold-up volume of solution in the in-line filter can be minimized by making filter design with lower hold up volumes or the dead volume can be compensated by overfill.

TABLE 6
Hold-up volume of in-line syringe filters

| Filter | Pore size | |
|---|---|---|
| Type | (μm) | Mean Hold-up Volume (μL) |
| PVDF | 0.45 | ~60 |
| PVDF | 0.20 | ~70 |

### Example 7: Determination of Instantaneous Force and Glide Force of Syringes with and without In-Line Syringe Filters

An universal testing machine operated by Nexygen Plus 3.0 software was used to determine the instantaneous and glide force. The syringes were filled with 0.5 ml Ranibizumab formulation butter. Three set of samples were studied.

Result: Force required to empty the contents of the syringe ranged between 5-6 Newton for 0.45 μm filter which is within the acceptable range.

TABLE 7
Break-loose and glide force of syringe in the presence
and absence of in-line syringe filters

| | Break-loose Force | Glide Force |
|---|---|---|
| Sample | (N) | (N) |
| Syringe (no filter) | 1.7 ± 0.2 | 1.7 ± 0.1 |
| Syringe with 0.45 μm PVDF in-line | 5.6 ± 0.5 | 5.9 ± 0.4 |
| syringe filter | | |

1. A syringe for administration of therapeutic protein or peptide comprising a syringe barrel, a stopper, a plunger and a needle with in-line filter wherein the therapeutic protein or peptide post-filtration from the syringe is substantially free of particulates with a diameter greater than 5 μm.

2. The syringe according to claim 1, wherein the therapeutic protein or peptide post-filtration from the syringe shows 85-99% reduction in particulates with a diameter of 2 μm as compared to a syringe without in-line filter.

3. The syringe according to claim 1, wherein the concentration of therapeutic protein or peptide post-filtration through syringe is similar to syringe without in-line filter.

4. The syringe according to claim 1, wherein the in-line filter has hold-up volume less than 500 μl.

5. The syringe according to claim 1 has an instantaneous force and glide force of less than about 6N.

6. The syringe according to claim 1 is a glass or plastic syringe with or without lubricant coating.

7. The syringe according to claim 1, wherein the syringe barrel has a coating of silicone oil from about 1 μg to about 800 μg per unit.

8. The syringe according to claim 1, wherein the syringe barrel has a coaling other than a silicone oil coating.

9. The syringe according to claim 1, wherein the in-line filter is made of polyethersulfone or polyvinyl difluoride or modified cellulose.

10. The syringe according to claim 1, wherein the in-line filter has pore size of about 0.1 μm to 10.0 μm.

11. The syringe according to claim 1 has been sterilized by steam, ethylene oxide or gamma radiation.

12. The syringe according to claim 1 has a maximum fill volume of between about 0.05 ml to about 5.0 ml.

13. The syringe according to claim 1, wherein the therapeutic protein or peptide includes monoclonal antibodies, fusion proteins, Fabs, Antibody-drag conjugates, bispecific antibodies, scFv, of synthetic, recombinant or plasma origin.

14. The syringe according to claim 1, wherein the therapeutic protein or peptide is a VEGF antagonist.

15. The syringe according to claim 14, wherein the VHGF antagonist is ranibizumab or aflibercept used for ocular diseases.

16. The syringe for the use according to claim 15, wherein the ocular disease is selected from the group consisting of age-related macular degeneration (AMD), visual impairment due to diabetic macular oedema (DME), visual impairment due to macular oedema secondary to retinal vein occlusion (branch RVO or central RVO), diabetic retinopathy in patients with diabetic inacular edema or visual impairment due to choroidal neovascularization (CNV) secondary to pathologic myopia.
